(19) United States
(12) Patent Application Publication
(10) Pub. No.: US 2011/0039311 A1
(43) Pub. Date: Feb. 17, 2011

(54) PROCESS FOR PRODUCING FERMENTATION PRODUCT FROM LIGNOCELLULOSE-CONTAINING MATERIAL

(75) Inventors: Xin Li, Raleigh, NC (US); Mads Peter Torry Smith, Raleigh, NC (US)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/919,572

(22) PCT Filed: Mar. 27, 2009
(86) PCT No.: PCT/US2009/038514

§ 371 Date: Aug. 26, 2010

Related U.S. Application Data
(60) Provisional application No. 61/039,823, filed on Mar. 27, 2008.

(51) Int. Cl. C12P 19/02; C12P 7/06

(52) U.S. Cl. 435/105; 435/161

(57) ABSTRACT

The invention relates to processes process of producing fermentation products from lignocellulose-containing material comprising: a) pretreating the lignocellulose-containing material; b) preparing a slurry of pretreated lignocellulose-containing material and thermo treated distiller's grain; c) hydrolyzing the slurry with one or more cellulolytic enzymes; d) fermenting with a fermenting organism.

### TECHNICAL FIELD

The present invention relates to processes of producing fermentation products from lignocellulose-containing material using a fermenting organism.

### BACKGROUND ART

Due to the limited reserves of fossil fuels and worries about emission of greenhouse gasses there is an increasing focus on using renewable energy sources. Commercial production of biofuels (mainly ethanol) and other fermentation products from starch and sugars is already ongoing, but the production cost is relatively high primarily because grains and sugar crops are expensive feedstocks. Therefore, the attention has turned towards the cheaper lignocellulose feedstocks (i.e., biomass) such as agricultural residues, grasses etc.

Processes for producing biofuels from lignocellulose-containing materials are described in the art and conventionally include the steps of pretreatment, hydrolysis, and fermentation. Lignocellulose-based processes are too expensive, so there is still a need for improving such processes.

### SUMMARY OF THE INVENTION

The first aspect of the invention relates to processes of producing fermentation products from lignocellulose-containing material, comprising:
a) pretreating lignocellulose-containing material;
b) preparing a slurry of pretreated lignocellulose-containing material and thermo treated distiller's grain;
c) hydrolyzing the slurry with one or more cellulolytic enzymes;
d) fermenting with a fermenting organism.

### BRIEF DESCRIPTION OF THE FIGURES

FIG. 1 shows the effect of DDG and enzyme dosage on hydrolysis of pre-treated corn stover (PCS).

FIG. 2 shows the effect of DDG and enzyme dosage on carbohydrate conversion rate of PCS.

FIG. 3 shows the effect on ethanol yield of added DDG before enzymatic hydrolysis.

### DETAILED DESCRIPTION OF THE INVENTION

### Distiller's Grain (DG)

Distiller's grain (DG) is a well known term used in the art and refers generally to de-alcoholized fermentation residues which remain after cereal grains have been fermented and distilled. Distiller's grain (DG) includes any of the typical grains, such as, but not limited to, corn, wheat, and rice, and is typically derived from dry mill fuel or beverage alcohol processes. During fermentation most starch is removed and the remaining distiller's grain is generally high in fiber, and contains dried yeast cells and highly digestible proteins.

More specifically, the starch-rich material is initially degraded to fermentable sugars by hydrolyzing enzymes (typically alpha-amylase and glucoamylase) which is converted directly or indirectly into the desired fermentation product using a suitable fermenting organism. Liquid fermentation products, such as ethanol, are typically recovered from the fermented mash (often referred to as “beer mash”), e.g., by distillation, which separates the desired fermentation product from other liquids and/or solids. The remaining faction, referred to as “whole stillage,” is dewatered and separated into a solid and a liquid phase, e.g., by centrifugation. The solid phase is referred to as “wet distiller's grain” (or “wet cake”) and the liquid phase (supernatant) is referred to as “thin stillage.” Wet distiller's grains (WDG) may be dried to provide “distiller's dried grain” (DDG) often used as nutrient in animal feed. Thin stillage is typically evaporated to provide condensate and syrup or may alternatively be recycled directly to a slurry tank as “backset.” Condensate may be forwarded to a methanator before being discharged or recycled to a slurry tank. The syrup consisting mainly of limit dextrins and non-fermentable sugars may be blended into DDG or added to the wet distiller's grains before drying to produce DDG/S (distillers dried grain with solubles).

The inventors found that when combining pre-treated corn stover with thermo treated distiller's dried grains (DDG) before hydrolysis, the sugar yield increased, the carbohydrate conversion rate was improved and a higher ethanol yield was obtained. Examples 1 and 2 provide further detail of these improvements. Additionally, the enzyme dosage used for hydrolysis may be reduced, resulting in overall reduced process costs.

Accordingly, in the first aspect the invention relates to processes of producing fermentation products from lignocellulose-containing material, comprising:
a) pretreating lignocellulose-containing material;
b) preparing a slurry of pretreated lignocellulose-containing material and thermo treated distiller's grain;
c) hydrolyzing the slurry with one or more cellulolytic enzymes;
d) fermenting with a fermenting organism.

The lignocellulose-containing material primarily consists of cellulose, hemicellulose, and lignin. Distiller's grain (DG) is defined above, but is preferably selected from distiller's dried grain (DDG), distiller's dried grains with solubles (DDG/S) and wet distillers' grain (WDG).

Themo treatment of distiller's grain according to the invention may be carried out using any suitable method. In an embodiment thermo treatment may be carried out, for instance, at above 60° C. for between 10 minutes and 48 hours, such as above 80° C. for between 5 minutes and 24 hours, such as at above 100° C. for 1 minute to 12 hour, such as around 121° C. for between 10 seconds and 6 hours, such as around 121° C. for between 10 and 30 minutes, such as around 15 minutes. For instance, thermo treatment may be done under pressure in an autoclave. One of ordinary skill in the art would be able to determine suitable thermo treatment conditions to open up the structure of distiller's grain. By opening up the structure of distiller's grain compounds capable of blocking lignin are released. Blocking of the lignin leads to reduced lignin inhibition of hydrolytic enzymes and/or the fermentation capacity of the fermenting organism. It is believed that the blocking compounds include peptides and lipids.

The pre-treated lignocellulose-containing material may be unwashed or washed, or may be a combination of washed and unwashed pre-treated lignocellulose-containing material. The material may be pre-treated lignocellulose-containing material that has been detoxified in any suitable way. The pre-treated lignocellulose-containing material may in an embodiment constitute from 5-30 wt. %, preferably from 10-25 wt. % of the slurry in step b).

The pre-treated lignocellulose-containing material may in an embodiment constitute from above 5 to 90 wt. %, preferably 10-80 wt. % of the total solids (TS) in the slurry in step b). Distiller's grain may in an embodiment constitute from above 0.1-40 wt. %, such as 1-30 wt. % of the pre-treated lignocellulose-containing material. TS% (i.e., pre-treated lignocellulose-containing material and distiller's grain) in step b) would normally lie in the range from 1-20 wt. %, preferably between 2-15 wt. %.

Examples of fermentation products other than ethanol can be found below in the section “Fermentation Products.”

In a preferred embodiment hydrolysis in step c) and fermentation in step d) are carried out as a simultaneous hydrolysis and fermentation process step (SSF process) or a hybrid hydrolysis and fermentation process step (HHF process). Hydrolysis, SSF or HHF may be carried out using cellulolytic enzymes or hemicellulolytic enzymes, or a combination thereof. However, other hydrolytic enzymes may also be present. Examples of cellulolytic enzymes, hemicellulolytic enzymes and other hydrolytic enzymes can be found in the “Enzymes”-section below.

The fermenting organism used in step d), SSF or HHF is typically of microbial origin, preferably yeast origin, preferably a strain of the genus Saccharomyces, Pichia, or Kluyveromyces. However, fermentation organisms of, e.g., bacterial origin are also contemplated. A non-exhaustive list of fermenting organisms can be found below in the section “Fermentation Organisms”. In a preferred embodiment the fermentation product is a biofuel, such as especially an alcohol, such as ethanol or butanol. Generally, fermentation or SSF is carried out at a temperature between 25° C. and 40° C., such as between 29° C. and 35° C., such as between 30° C. and 34° C., such as around 32° C. The pH typically is in the pH between 3 and 8, preferably between 4 and 6. Further fermentations may be carried out for between 1-120 hours, preferably between 8-96 hours.

### Hydrolysis

According to the invention the pre-treated lignocellulose-containing material is hydrolyzed together with thermo treated distiller's grain in an aqueous slurry. In a preferred embodiment hydrolysis is carried out enzymatically using a hydrolytic enzyme or mixture of hydrolytic enzymes. According to the invention the pretreated lignocellulose-containing material, to be fermented, is hydrolyzed by one or more hydrolases (class EC 3 according to the Enzyme Nomenclature), preferably one or more carbohydrases selected from the group consisting of cellulase, hemicellulase, or amylase, such as alpha-amylase, maltogenic amylase or beta-amylase. A protease may also be present.

The enzyme(s) used for hydrolysis are capable of directly or indirectly converting carbohydrate polymers (e.g., cellulose and/or hemicellulose) into fermentable sugars which can be fermented into a desired fermentation product, such as ethanol.

In a preferred embodiment the carbohydrase has cellulolytic enzyme activity. Suitable carbohydrases are described in the “Enzymes” section below.

Hemicellulose polymers can be broken down by hemicellulases and/or acid hydrolysis to release its five and six carbon sugar components. The six carbon sugars (hexoses), such as glucose, galactose and mannose, can readily be fermented to, e.g., ethanol, acetone, butanol, glycerol, citric acid, fumaric acid etc. by suitable fermenting organisms including yeast. Preferred for ethanol fermentation is yeast of the species Saccharomyces cerevisiae, preferably strains which are resistant towards high levels of ethanol, i.e., up to, e.g., about 10, 12, 15 or 20 vol. % or more ethanol.

In a preferred embodiment the lignocellulose-containing material is hydrolyzed using a hemicellulase, preferably a xylanase, esterase, cellobiase, or combination of two or more thereof.

Hydrolysis may also be carried out in the presence of a combination of hemicellulases and/or cellulases, and optionally one or more of the other enzyme activities mentioned above.

The enzymatic treatment may be carried out in a suitable aqueous environment under conditions which can readily be determined by one skilled in the art. In a preferred embodiment hydrolysis is carried out at optimal conditions for the enzyme(s) in question.

Suitable process time, temperature and pH conditions can readily be determined by one skilled in the art. Preferably, hydrolysis is carried out at a temperature between 20 and 70° C., preferably between 25 and 60° C., especially around 50° C. Hydrolysis is preferably carried out at a pH in the range from 4-8, preferably pH 5-7. Preferably, hydrolysis is carried out for between 6 and 96 hours, preferably between 12 and 48 hours, especially around 24 hours.

### Fermentation of Lignocellulose Derived Material

Fermentation of lignocellulose-containing material may be carried out in any suitable way. Suitable conditions depend on the fermenting organism, the substrate and the desired product. One skilled in the art can easily determine what suitable fermentation conditions are. Examples of suitable conditions are given above and below. According to the invention hydrolysis in step c) and fermentation in step d) may be carried out simultaneously (SSF process) or as a hybrid process (HHF process).

### Lignocellulose-Containing Material (Biomass)

Any suitable lignocellulose-containing material is contemplated in context of the present invention. Lignocellulose-containing material may be any material containing lignocellulose. In a preferred embodiment the lignocellulose-containing material contains at least 50 wt. %, preferably at least 70 wt. %, more preferably at least 90 wt. % lignocellulose. It is to be understood that the lignocellulose-containing material may also comprise other constituents such as cellulosic material, such as cellulose, hemicellulose and may also comprise constituents such as sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulosic material can also be, but is not limited to, herbaceous material, agricultural residues, forestry residues, municipal solid wastes, waste paper, and pulp and paper mill residues. It is understood herein that lignocellulose-containing material may be in the form of plant cell wall material containing lignin, cellulose, and hemi-cellulose in a mixed matrix.

In an embodiment the lignocellulose-containing material is corn fiber, rice straw, pine wood, wood chips, bagasse, paper and pulp processing waste, corn stover, corn cobs, hardwood such as poplar and birch, softwood, cereal straw such as wheat straw, switch grass, miscanthus, rice hulls, municipal solid waste (MSW), industrial organic waste, office paper, or mixtures thereof.

In a preferred embodiment the lignocellulose-containing material is corn stover or corn cobs. In another preferred embodiment, the lignocellulose-containing material is corn fiber. In another preferred embodiment, the lignocellulose-containing material is switch grass. In another preferred embodiment, the the lignocellulose-containing material is bagasse.

### SSF, HHF and SHF

In one embodiment of the present invention, hydrolysis and fermentation is carried out as a simultaneous hydrolysis and fermentation step (SSF). In general this means that combined/simultaneous hydrolysis and fermentation are carried out at conditions (e.g., temperature and/or pH) suitable, preferably optimal, for the fermenting organism(s) in question.

In another embodiment hydrolysis step and fermentation step are carried out as hybrid hydrolysis and fermentation (HHF). HHF typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable, preferably optimal, for the hydrolyzing enzyme(s) in question. The subsequent simultaneous hydrolysis and fermentation step is typically carried out at conditions suitable for the fermenting organism(s) (often at lower temperatures than the separate hydrolysis step).

In another embodiment, the hydrolysis and fermentation steps may also be carried out as separate hydrolysis and fermentation, where the hydrolysis is taken to completion before initiation of fermentation. This is often referred to as “SHF.”

### Fermenting Organisms

The term “fermenting organism” refers to any organism, including bacterial and fungal organisms, including yeast and filamentous fungi, suitable for producing a desired fermentation product. The fermenting organism may be C6 or C5 fermenting organisms, or a combination thereof. Both C6 and C5 fermenting organisms are well known in the art.

Suitable fermenting organisms according to the invention are able to ferment, i.e., convert fermentable sugars, such as glucose, fructose maltose, xylose, mannose or arabinose, directly or indirectly into the desired fermentation product. Examples of fermenting organisms include fungal organisms such as yeast. Preferred yeast includes strains of the genus Saccharomyces, in particular strains of Saccharomyces cerevisiae or Saccharomyces uvarum; a strain of Pichia, preferably Pichia stipitis such as Pichia stipitis CBS 5773 or Pichia pastoris; a strain of the genus Candida, in particular a strain of Candida utilis, Candida arabinofermentans, Candida diddensii, Candida sonorensis, Candida shehatae, Candida tropicalis, or Candida boidinii. Other fermenting organisms include strains of Hansenula, in particular Hansenula polymorpha or Hansenula anomala; Kluyveromyces, in particular Kluyveromyces fragilis or Kluyveromyces marxianus; and Schizosaccharomyces, in particular Schizosaccharomyces pombe.

Preferred bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes and strains of Thermoanaerobacter, in particular Thermoanaerobacter BG1L1 (Appl. Microbiol. Biotech. 77: 61-86) and Thermoanarobacter ethanolicus, Thermoanaerobacter thermosaccharolyticum, or Thermoanaerobacter mathranii. Strains of Lactobacillus are also envisioned as are strains of Corynebacterium glutamicum R, Bacillus thermoglucosidaisus, and Geobacillus thermoglucosidasius.

In an embodiment the fermenting organism is a C6 sugar fermenting organism, such as a strain of, e.g., Saccharomyces cerevisiae.

In connection with fermentation of lignocellulose derived materials, C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp. that are capable of fermenting C5 sugars include the ones concerned in, e.g., Ho et al., 1998, Applied and Environmental Microbiology, p. 1852-1859 and Karhumaa et al., 2006, Microbial Cell Factories 5:18, and Kuyper et al., 2005, FEMS Yeast Research 5: 925-934.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

According to the invention the fermenting organism capable of producing a desired fermentation product from fermentable sugars, including glucose, fructose maltose, xylose, mannose, and/or arabinose, is preferably grown under precise conditions at a particular growth rate. When the fermenting organism is introduced into/added to the fermentation medium the inoculated fermenting organism pass through a number of stages. Initially growth does not occur. This period is referred to as the “lag phase” and may be considered a period of adaptation. During the next phase referred to as the “exponential phase” the growth rate gradually increases. After a period of maximum growth the rate ceases and the fermenting organism enters “stationary phase”. After a further period of time the fermenting organism enters the “death phase” where the number of viable cells declines.

### Fermentation Products

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes. The fermentation product, such as ethanol, obtained according to the invention, may preferably be used as biofuel. However, in the case of ethanol it may also be used as potable ethanol.

### Fermentation of Lignocellulose-Derived Sugars

As mentioned above, different kinds of fermenting organisms may be used for fermenting sugars derived from lignocellulose-containing materials. Fermentations are typically carried out by yeast, bacteria or filamentous fungi, including the ones mentioned in the “Fermenting Organisms” section above. If the aim is C6 fermentable sugars the conditions are usually similar to the well known starch fermentation conditions. However, if the aim is to ferment C5 sugars (e.g., xylose) or a combination of C6 and C5 fermentable sugars the fermenting organism(s) and/or fermentation conditions may differ.

Bacteria fermentations may be carried out at higher temperatures, such as up to 75° C., e.g., between 40-70° C., such as between 50-60° C., than conventional yeast fermentations, which are typically carried out at temperatures from 20-40° C. However, bacteria fermentations at temperatures as low as 20° C. are also known. Fermentations are typically carried out at a pH in the range between 3 and 7, preferably from pH 3.5 to 6, such as around pH 5. Fermentations are typically ongoing for 24-96 hours.

### Recovery

Subsequent to fermentation the fermentation product may be separated from the fermented slurry. The slurry may be distilled to extract the desired fermentation product or the desired fermentation product may be extracted from the fermented slurry by micro or membrane filtration techniques. Alternatively the fermentation product may be recovered by stripping. Methods for recovery are well known in the art.

### Enzymes

Even if not specifically mentioned in context of a process of the invention, it is to be understood that the enzyme(s) are used in an effective amount.

### Cellulolytic Enzymes

One or more cellulolytic enzymes may be present during fermentation, hydrolysis, SSF or HHF.

The terms “cellulolytic enzymes” as used herein are understood as comprising the cellobiohydrolases (EC 3.2.1.91), e.g., cellobiohydrolase I and cellobiohydrolase II, as well as the endo-glucanases (EC 3.2.1.4) and beta-glucosidases (EC 3.2.1.21). See relevant sections below with further description of such enzymes. In order to be efficient, the digestion of cellulose may require several types of enzymes acting cooperatively. At least three categories of enzymes are often needed to convert cellulose into glucose: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

The cellulolytic enzyme may comprise a carbohydrate-binding module (CBM) which enhances the binding of the enzyme to a lignocellulose-containing fiber and increases the efficacy of the catalytic active part of the enzyme. A CBM is defined as contiguous amino acid sequence within a carbohydrate-active enzyme with a discreet fold having carbohydrate-binding activity. For further information of CBMs see the CAZy internet server (Supra) or Tomme et al. (1995) in Enzymatic Degradation of Insoluble Polysaccharides (Saddler and Penner, eds.), Cellulose-binding domains: classification and properties. pp. 142-163, American Chemical Society, Washington.

In a preferred embodiment the cellulases or cellulolytic enzymes may be a cellulolytic preparation as defined PCT/2008/065417, which is hereby incorporated by reference. In a preferred embodiment the cellulolytic preparation comprising a polypeptide having cellulolytic enhancing activity (GH61A), preferably the one disclosed in WO 2005/074656. The cellulolytic preparation may further comprise a beta-glucosidase, such as a beta-glucosidase derived from a strain of the genus Trichoderma, Aspergillus or Penicillium, including the fusion protein having beta-glucosidase activity disclosed in WO2008/057637 (Novozymes). In an embodiment the cellulolytic preparation may also comprises a CBH II, preferably Thielavia terrestris cellobiohydrolase II (CEL6A). In an embodiment the cellulolytic preparation also comprises a cellulase enzymes preparation, preferably the one derived from Trichoderma reesei or Humicola insolens.

The cellulolytic activity may, in a preferred embodiment, be derived from a fungal source, such as a strain of the genus Trichoderma, preferably a strain of Trichoderma reesei; or a strain of the genus Humicola, such as a strain of Humicola insolens; or a strain of Chrysosporium, preferably a strain of Chrysosporium lucknowense.

In an embodiment the cellulolytic enzyme preparation comprises a polypeptide having cellulolytic enhancing activity (GH61A) disclosed in WO 2005/074656; a cellobiohydrolase, such as Thielavia terrestris cellobiohydrolase II (CEL6A), a beta-glucosidase (e.g., the fusion protein disclosed in WO 2008/057634) and cellulolytic enzymes, e.g., derived from Trichoderma reesei.

In an embodiment the cellulolytic enzyme preparation comprises a polypeptide having cellulolytic enhancing activity (GH61A) disclosed in WO 2005/074656; a beta-glucosidase (e.g., the fusion protein disclosed in WO 2008/057637) and cellulolytic enzymes, e.g., derived from Trichoderma reesei.

In an embodiment the cellulolytic enzyme composition is the commercially available product CELLUCLAST™ 1.5L, CELLUZYME™ (from Novozymes NS, Denmark) or ACCELERASE™ 1000 (from Genencor Inc. USA).

A cellulase may be added for hydrolyzing the pre-treated lignocellulose-containing material. The cellulase may be dosed in the range from 0.1-100 FPU per gram total solids (TS), preferably 0.5-50 FPU per gram TS, especially 1-20 FPU per gram TS. In another embodiment at least 0.1 mg cellulolytic enzyme per gram total solids (TS), preferably at least 3 mg cellulolytic enzyme per gram TS, such as between 5 and 10 mg cellulolytic enzyme(s) per gram TS is(are) used for hydrolysis.

### Endoglucanase (EG)

Endoglucanases (EC No. 3.2.1.4) catalyze endo hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification. Endoglucanase activity may be determined using carboxymethyl cellulose (CMC) hydrolysis according to the procedure of Ghose, 1987, Pure and Appl. Chem. 59: 257-268.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

### Cellobiohydrolase (CBH)

The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydroloses are mentioned above including CBH I and CBH II from Trichoderma reseei; Humicola insolens and CBH II from Thielavia terrestris cellobiohydrolase (CELL6A)

Cellobiohydrolase activity may be determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288. The Lever et al. method is suitable for assessing hydrolysis of cellulose in corn stover and the method of van Tilbeurgh et al. is suitable for determining the cellobiohydrolase activity on a fluorescent disaccharide derivative.

### Beta-Glucosidase

One or more beta-glucosidases (sometimes referred to as “cellobiases”) may be present during hydrolysis, fermentation, SSF or HHF.

The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42: 55-66, except different conditions were employed as described herein. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

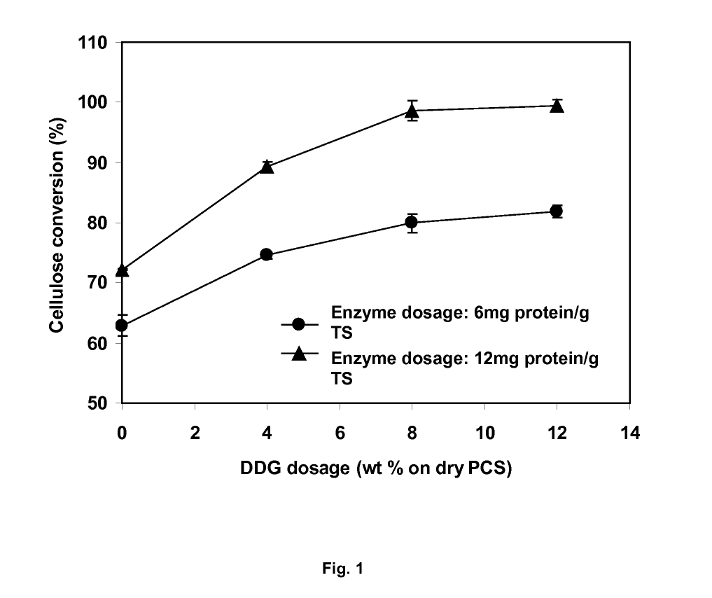

In a preferred embodiment the beta-glucosidase is of fungal origin, such as a strain of the genus Trichoderma, Aspergillus or Penicillium. In a preferred embodiment the beta-glucosidase is a derived from Trichoderma reesei, such as the beta-glucosidase encoded by the bgl1 gene (see FIG. 1 of EP 562003). In another preferred embodiment the beta-glucodidase is derived from Aspergillus oryzae (recombinantly produced in Aspergillus oryzae according to WO 02/095014), Aspergillus fumigatus (recombinantly produced in Aspergillus oryzae according to Example 22 of WO 02/095014) or Aspergillus niger (1981, J. Appl. 3: 157-163).

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Application Publication No. 2007/0077630 discloses an isolated polypeptide having cellulolytic enhancing activity and a polynucleotide thereof from Trichoderma reesei.

### Hemicellulolytic Enzymes

Hemicellulose can be broken down by hemicellulases and/or acid hydrolysis to release its five and six carbon sugar components.

In an embodiment of the invention the lignocellulose derived material may be treated with one or more hemicellulases.

Any hemicellulase suitable for use in hydrolyzing hemicellulose, preferably into xylose, may be used. Preferred hemicellulases include xylanases, arabinofuranosidases, acetyl xylan esterase, feruloyl esterase, glucuronidases, galactanase, endo-galactanase, mannases, endo or exo arabinases, exo-galactanses, pectinase, xyloglucanase, or mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes NS, Denmark).

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes NS, Denmark.

The hemicellulase may be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

Arabinofuranosidase (EC 3.2.1.55) catalyzes the hydrolysis of terminal non-reducing alpha-L-arabinofuranoside residues in alpha-L-arabinosides.

Galactanase (EC 3.2.1.89), arabinogalactan endo-1,4-beta-galactosidase, catalyzes the endohydrolysis of 1,4-D-galactosidic linkages in arabinogalactans.

Pectinase (EC 3.2.1.15) catalyzes the hydrolysis of 1,4-alpha-D-galactosiduronic linkages in pectate and other galacturonans.

Xyloglucanase catalyzes the hydrolysis of xyloglucan.

The hemicellulase may be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

### Xylose Isomerase

Xylose isomerases (D-xylose ketoisomerase) (E.C. 5.3.1.5.) are enzymes that catalyze the reversible isomerization reaction of D-xylose to D-xylulose. Some xylose isomerases also convert the reversible isomerization of D-glucose to D-fructose. Therefore, xylose isomarase is sometimes referred to as “glucose isomerase.”

A xylose isomerase used in a method or process of the invention may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus, Flavobacterium, and Thermotoga, including T. neapolitana (Vieille et al., 1995, Appl. Environ. Microbiol. 61(5): 1867-1875) and T. maritime.

Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., 1988, Agric. Biol. Chem. 52(7): 1817-1824. The xylose isomerase may preferably be derived from a strain of Candida boidinii (Kloeckera 2201), deposited as DSM 70034 and ATCC 48180, disclosed in Ogata et al., Agric. Biol. Chem. 33: 1519-1520 or Vongsuvanlert et al., 1988, Agric. Biol. Chem. 52(2): 1519-1520.

In one embodiment the xylose isomerase is derived from a strain of Streptomyces, e.g., derived from a strain of Streptomyces murinus (U.S. Pat. No. 4,687,742); S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Pat. No. 4,351,903, U.S. Pat. No. 4,137,126, U.S. Pat. No. 3,625,828, HU patent no. 12,415, DE patent 2,417,642, JP patent no. 69,28,473, and WO 2004/044129 each incorporated by reference herein.

The xylose isomerase may be either in immobilized or liquid form. Liquid form is preferred.

Examples of commercially available xylose isomerases include SWEETZYME™ T from Novozymes NS, Denmark.

The xylose isomerase is added to provide an activity level in the range from 0.01-100 IGIU per gram total solids.

### Other Enzymes

Other hydrolytic enzymes may also be present during hydrolysis, fermentation, SSF or HHF. Contemplated enzymes include alpha-amylases; glucoamylases or another carbohydrate-source generating enzymes, such as beta-amylases, maltogenic amylases and/or alpha-glucosidases; proteases; or mixtures of two of more thereof.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

### MATERIALS & METHODS

### Materials

### Cellulolytic Preparation A:

Cellulolytic composition comprising a polypeptide having cellulolytic enhancing activity (GH61A) disclosed in WO 2005/074656; a beta-glucosidase (fusion protein disclosed in WO 2008/057637), and cellulolytic enzymes preparation derived from Trichoderma reesei. Cellulase preparation A is disclosed in co-pending application PCT/US2008/065417.

Yeast: RED START™ available from Red Star/Lesaffre, USA
Dilute acid pretreated corn stover (PCS) used in Examples 1 and 2 was obtained from NREL, USA.
DDG used in Examples 1 and 2 was obtained from ADKINS ENERGY LLC, USA.

### Equipment:

Autoclave used in Examples 1 and 2 is from Tuttnauer USA Co., Hauppauge, N.Y. 11788 USA Model: 3870 EA, Serious No. 2304643)

### Methods

### Measurement of Cellulase Activity Using Filter Paper Assay (FPU Assay)

### 1. Source of Method

1.1 The method is disclosed in a document entitled “Measurement of Cellulase Activities” by Adney and Baker, 1996, Laboratory Analytical Procedure, LAP-006, National Renewable Energy Laboratory (NREL). It is based on the IUPAC method for measuring cellulase activity (Ghose, 1987, Measurement of Cellulase Activities, Pure & Appl. Chem. 59: 257-268.

### 2. Procedure

2.1 The method is carried out as described by Adney and Baker, 1996, supra, except for the use of a 96 well plates to read the absorbance values after color development, as described below.

### 2.2 Enzyme Assay Tubes:

2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.

### 2.4 Glucose Standards

2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:
G1=1.0 mL stock+0.5 mL buffer=6.7 mg/mL=3.3 mg/0.5 mL
G2=0.75 mL stock+0.75 mL buffer=5.0 mg/mL=2.5 mg/0.5 mL
G3=0.5 mL stock+1.0 mL buffer=3.3 mg/mL=1.7 mg/0.5 mL
G4=0.2 mL stock+0.8 mL buffer=2.0 mg/mL=1.0 mg/0.5 mL
   2.4.3 Glucose standard tubes are prepared by adding 0.5 mL of each dilution to 1.0 mL of citrate buffer.
   2.4.4 The glucose standard tubes are assayed in the same manner as the enzyme assay tubes, and done along with them.

### 2.5 Color Development

2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH₂O in a 96-well plate. Each well is mixed, and the absorbance is read at 540 nm.
2.6 Calculations (examples are given in the NREL document)
2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows:
FPU/mL=0.37/ enzyme dilution producing 2.0 mg glucose

### PHBA Assay

Reducing sugars react with hydrazides of benzoic acids in an alkaline medium to give the bis-benzoylhydrazones of glyoxal and methylglyoxal, both of which have intense yellow color. p-Hydroxybenzoic acid hydrazide (PHBAH) is utilized as the reactive hydrazide for the photometric determination of reducing sugars. The rate of the reaction is accelerated by the catalytic influence of bismuth ions, thereby increasing the sensitivity of the assay at lower temperatures. The generation of the bis-benzoylhydrazones of glyoxal and methylglyoxal is monitored at 405 nm and is proportional to the amount of reducing sugar concentration and, ultimately, carbohydrase activity on a polysaccharide substrate. The concentration of reducing sugar in an unknown sample is determined from a standard curve constructed from glucose calibration standards.

A detailed description (EUS-SM-0006.02/01) is available on request from Novozymes

### EXAMPLES

### Example 1

### Effect of DDG on Glucose Yield During Enzymatic Hydrolysis

Dilute acid pretreated corn stover (PCS) was washed with hot tap water before enzymatic hydrolysis. DDG was thermo treated at 121° C. for 15 minutes using an autoclave (Tuttnauer YSA co, NY). Thermo treated DDG (4, 8 or 12 wt. % dry PCS), and untreated DDG (control), respectively, was added to the washed PCS slurry (10 wt. % TS) and mixed. After 30 minutes, the PCS-DDG mixture was hydrolyzed at pH 4.8, 50° C. for 72 hours with

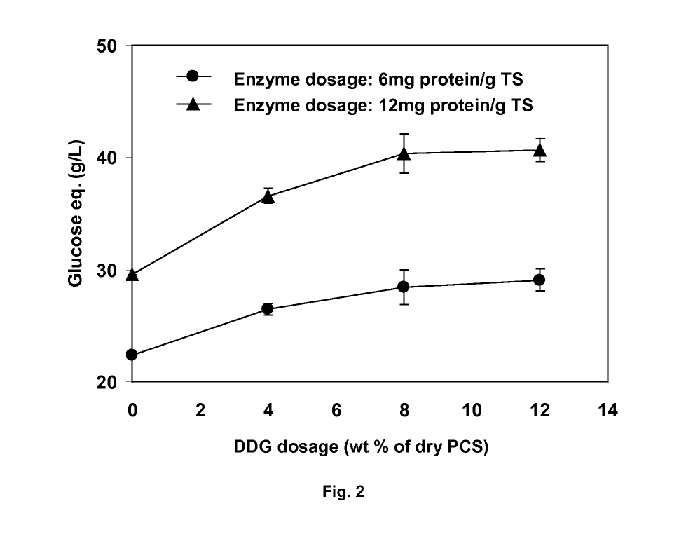

Cellulolytic Preparation A (6 mg protein/g TS or 12 mg protein/g TS). The content of released sugars was determined by PHBA method and confirmed by HPLC. After 72 hours hydrolysis, the final sugar yield increased from 29.5 g/L to 40.0 g/L with addition of thermo treated DDG (FIG. 2) at the enzyme dosage of 12 mg protein/g TS. The cellulose conversion of PCS was improved from 72% to 99% with addition of thermo treated DDG (FIG. 1) at the enzyme dosage of 12 mg protein/g TS. When the control (untreated DDG) was hydrolyzed under the same condition no additional sugars were released.

### Example 2

### Effect of DDG on fermentation and ethanol yield during enzymatic hydrolysis

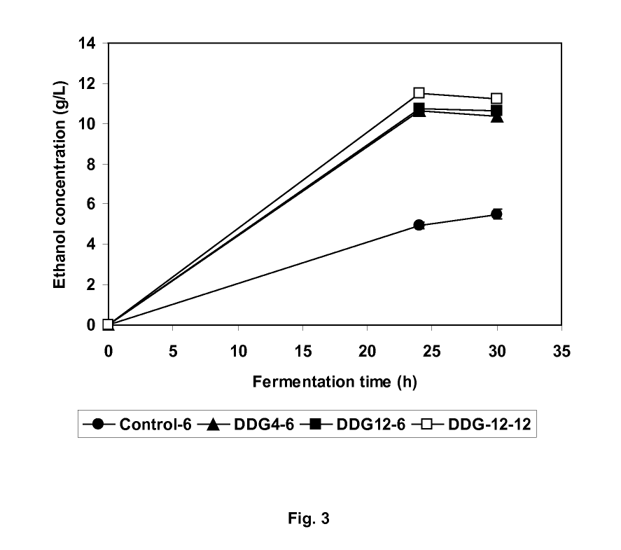

The same slurry of hydrolyzed pretreated corn stover (5 wt. % TS) and thermo treated DDG used in Example 1 was used for this experiment. After enzymatic hydrolysis, residues were removed by filtration and the hydrolysate was transferred to a fermentation tube. RED STAR™ yeast cells (0.25 g/L) was inoculated in the fermentation mixture without nutrition and incubated at 30° C., pH 5, 150 rpm. The content of sugar and ethanol was determined by HPLC. After fermentation, the ethanol content was analyzed by HPLC (FIG. 3). The addition of thermo treated DDG during enzymatic hydrolysis increased the fermentation speed and the ethanol yield from 5.5 g/L (no DDG added) to 11.5 g/L after 24 hours fermentation. The amount of DDG and enzyme are indicated for each sample. For example, “DDG 4-6” indicates that DDG in the amount of 4 wt. % PCS was used, and enzyme was dosed at 6 mg protein/g TS.

1-20. (canceled)

21. A process of producing a fermentation product from lignocellulose-containing material, comprising:
a) pretreating lignocellulose-containing material;
b) preparing a slurry of pretreated lignocellulose-containing material and thermo treated distiller's grain;
c) hydrolyzing the slurry with one or more cellulolytic enzymes; and
d) fermenting with a fermenting organism.

22. The process of claim 21, wherein the distiller's grain is selected from the group consisting of distiller's dried grains, distiller's dried grain with solubles, and wet distillers' grain.

23. The process of claim 21, wherein the themo treatment of distiller's grains is carried out at above 100° C. for 1 minute to 1 hour.

24. The process of claim 21, wherein the hydrolysis in step c) and fermentation in step d) is carried out as hybrid hydrolysis and fermentation steps or simultaneous hydrolysis and fermentation steps.

25. The process of claim 21, wherein the lignocellulose-containing material originates from materials selected from the group consisting of corn fiber, rice straw, wheat straw, pine wood, wood chips, bagasse, paper or pulp processing waste, corn stover, corn cobs, hardwood, softwood, cereal straw, switch grass, miscanthus, rice hulls, municipal solid waste, industrial organic waste, office paper, and mixtures thereof.

26. The process of claim 21, wherein the lignocellulose-containing material is chemically, mechanically or biologically pre-treated in step a).

27. The process of claim 21, wherein the pre-treated lignocellulose-containing material is unwashed or washed, or a combination of washed and unwashed pretreated lignocellulose-containing material, or is detoxified pre-treated lignocellulose-containing material.

28. The process of claim 21, wherein the pre-treated lignocellulose-containing constitutes from above 5 to 90 wt. %, of the TS in the slurry in step b).

29. The process of claim 28, wherein the distiller's grain constitutes from above 0 to 40 wt. % of the pre-treated lignocellulose-containing material.

30. The process of claim 21, wherein the cellulolytic enzymes used for hydrolysis of the pre-treated material includes one or more cellulases.

31. The process of claim 21, wherein the cellulolytic enzyme(s) used for hydrolysis is a cellulolytic preparation derived from a strain of Chrysosporium, Humicola, or Trichoderma.

32. The process of claim 21, further wherein one or more beta-glucosidases are present during hydrolysis, fermentation, HHF, or SSF.

33. The process of claim 32, wherein the beta-glucosidase is derived from a strain of Aspergillus, Chrysosporium, Humicola, or Trichoderma.

34. The process of claim 21, further wherein one or more polypeptides having cellulolytic enhancing activity are present during hydrolysis, fermentation, HHF or SSF.

35. The process of claim 34, wherein the polypeptide having cellulolytic enhancing activity is a family GH61A polypeptide derived from a strain of Thermoascus, Thielavia, or Trichoderma.

36. The process of claim 21, further comprising subjecting the material to one or more hemicellulolytic enzymes during hydrolysis, fermentation, HHF, or SSF.

37. The process of claim 21, wherein the fermenting organism is a C6 or C5 fermenting organism.

38. The process of claim 21, wherein the fermenting organism is a yeast.

39. The process of claim 21, wherein the fermentation product is an alcohol.
